(19) United States
(12) Patent Application Publication
(10) Pub. No.: US 2021/0220184 A1
(43) Pub. Date: Jul. 22, 2021

(54) ABSORBENT TAMPON FOR TREATMENT OF MENSTRUAL SYMPTOMS

(71) Applicant: Anne's Day Ltd, London (GB)

(72) Inventor: Valentina Milanova, London (GB)

(21) Appl. No.: 17/265,517

(22) PCT Filed: Aug. 2, 2019
(86) PCT No.: PCT/IB2019/056614

§ 371 Date: Feb. 2, 2021

Related U.S. Application Data
(60) Provisional application No. 62/714,656, filed on Aug. 3, 2018.

(51) Int. Cl. A61F 13/20; A61L 15/40; A61L 15/44; A61L 15/34

(52) U.S. Cl. CPC A61F 13/202; A61L 15/34; A61L 15/44; A61L 15/40

(57) ABSTRACT

A catamenial tampon for absorbing menstrual flow and for treating menstrual symptoms is disclosed. The tampon has an absorbent tampon body made of industrial hemp fiber. The tampon body is provided with a pharmaceutical composition comprising an effective amount of cannabidiol. Further disclosed is a method for making the tampon and a method for treatment of symptoms associated with menstruation using the tampon.

### BACKGROUND

Every month women are confronted with a plethora of problems associated with their menstrual period. These problems include nausea, headache, backache, cramping, bloating, and, occasionally, a very heavy flow. The common approach to address these problems is on one hand to utilize a catamenial tampon or sanitary napkin for absorption of the body fluids generally discharged during menstruation, and on the other hand to separately administer medication to alleviate pain and discomfort. The existing approach to manage menstrual flow and menstrual symptoms has several drawbacks.

Existing catamenial tampons utilize rayon, viscose and other petrol-derived fibers, which are detrimental to human health, especially when used in the most absorbent part of the human body, the vagina. In addition, tampon materials are bleached with toxic chemicals, including artificial colors, polyester, adhesives, polyethylene, polypropylene, and propylene glycol. These are linked to hormone disruption, cancer, vaginal dryness, and infertility.

In addition, oral administration of pain-relieving medication can have negative side effects, e.g. stomach and GI track problems due to ingestion of ibuprofen or paracetamol. Furthermore, nausea and vomiting are common symptoms associated with menstruation and may also reduce the amount of orally administered medication and thus the effective amount available to establish sufficient levels of the medication in the circulation of the subject.

It is therefore an objective of an embodiment to provide an improved tampon which increases comfort and utility levels of consumers and is also more environmentally friendly.

### SUMMARY OF THE INVENTION

According to one embodiment a tampon includes an absorbent tampon body made of industrial hemp fiber, wherein the tampon body is provided with a pharmaceutical composition comprising an effective amount of cannabidiol.

In an embodiment, the tampon overcomes the problems of prior art approaches to manage menstrual flow and symptoms associated with menstruation. Using industrial hemp fiber as material for the tampon body provides the tampon with superior absorbance properties for absorbing menstrual flow. Furthermore, tests performed by the inventors have shown that cannabidiol can be delivered vaginally when provided on the tampon to treat symptoms associated with menstruation, such as headaches, cramps, bloating, nausea and vomiting.

The tampon thus may function both for absorbing menstrual flow with high efficiency and at the same time for delivering a pharmaceutical agent in the form of cannabidiol. The tampon therefore eliminates the need to separately orally administer medication to alleviate menstrual symptoms, a step which is required in prior art solutions to manage menstrual symptoms. As a result, women will also be spared the common negative side effects of taking pain-relieving medication orally, e.g. stomach and GI track problems due to ingestion of for example ibuprofen or paracetamol.

Since CBD is released in the vaginal canal, where a pH of 3.8-4.5 is normal, CBD will not be destroyed in such an environment. The CBD will absorb across the mucous lining on the vaginal wall and enter the bloodstream directly. As a result, the amount of CBD remains higher compared to oral administration where the amount of active agent is reduced for example due to first pass liver metabolism.

In an embodiment, the tampon is also more environmentally friendly than prior art products. Organic industrial hemp is grown without the need for pesticides, herbicides, and fertilizers, making the crop truly organic and thus safer to use inside the female body. Hemp requires no irrigation, apart from rainwater, meaning the crop has no environmental footprint. Industrial hemp is also biodegradable, which means users can flush the used product, without worrying about pollution.

In an embodiment, a method for producing an absorbent tampon for delivering a therapeutic agent includes the steps of manufacturing an absorbent tampon having a tampon body made from industrial hemp fiber; and providing the tampon body with a pharmaceutical composition comprising an effective amount of cannabidiol.

### BRIEF DESCRIPTION OF THE DRAWINGS

Other features and advantages of the present invention will be more readily apparent upon reading the following description of currently preferred exemplified embodiments of the invention with reference to the accompanying drawing, in which:

FIG. 1 is a perspective view of an embodiment of a tampon;

FIG. 2 is a perspective view of another embodiment of the tampon;

FIG. 3 is a diagram of the tampon shown in FIG. 1 with a CBD capsule;

FIG. 4 is a diagram of the tampon shown in FIG. 2 with a CBD capsule;

FIG. 5 is a vertical cross-sectional view of an embodiment of the tampon with a CBD capsule;

FIG. 6 is a horizontal cross-sectional view of an embodiment of the tampon;

FIG. 7 is a perspective view of a tampon with a CBD strip applied on the tampon body;

FIG. 8 is a perspective view of another embodiment of a tampon with a CBD strip applied on the tampon body;

FIG. 9 is a perspective view of another embodiment of a tampon with a CBD tip applied on the tampon body;

FIG. 10 is a perspective view of another embodiment of a tampon with a CBD tip applied on the tampon body;

FIG. 11 is a schematic illustration of an exemplary method of producing a tampon by rolling;

FIG. 12 is a schematic illustration of an exemplary method of producing a tampon by folding;

FIG. 13 is a perspective view of an embodiment of a tampon showing placement of a protective sock around the body of the tampon;

FIG. 14 is a perspective view of an embodiment of a tampon illustrating closure of the protective sock by sewing; and

FIG. 15 is a perspective view of an embodiment of a finished tampon.

### DETAILED DESCRIPTION OF THE PREFERRED EMBODIMENTS

Throughout all the figures, same or corresponding elements may generally be indicated by same reference numerals. These depicted embodiments are to be understood as illustrative of the invention and not as limiting in any way. It should also be understood that the figures are not necessarily to scale and that the embodiments may be illustrated by graphic symbols, phantom lines, diagrammatic representations and fragmentary views. In certain instances, details which are not necessary for an understanding of the present invention or which render other details difficult to perceive may have been omitted.

As used herein, the term “proximal end” means the end of the tampon body situated closest to the cervix of the vagina when the tampon, is inserted therein.

As used herein “distal end” means the end of the tampon body situated away from the uterus and closest to the vaginal introitus.

As used herein “effective amount of cannabidiol” means an amount of cannabidiol that is sufficient to inhibit the occurrence or ameliorate one or more symptoms associated with menstruation.

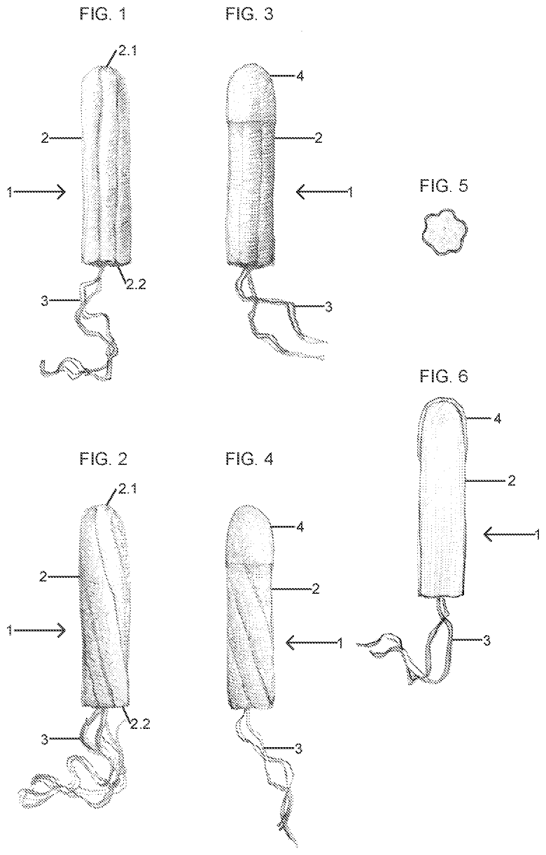

Turning now to the drawing, and in particular to FIG. 1, there is shown a perspective view of a tampon 1 according to an embodiment. The tampon 1 has a tampon body 2 and a withdrawal string 3 for withdrawal of the tampon 1 from the vaginal cavity after use. The tampon 1 has a proximal end 2.1 that is closer to the cervix when the tampon 1 is in use. The tampon 1 also has a distal end 2.2 which is closer to the vaginal introitus when the tampon 1 is in use. The tampon 1 generally has a cylindrical shape and can have a rounded or blunt distal or proximal end. Narrow channels in the tampon 1 may include cannabidiol (in the following also referred to as CBD).

FIG. 2 shows a perspective view of another embodiment of tampon 1 where the channels including cannabidiol are diagonal from the proximal end 2.1 to the distal end 2.2 of the tampon 1.

The tampon 1 is provided with a pharmaceutical composition comprising cannabidiol. The tampon 1 can be provided with the pharmaceutical composition in various ways. In an embodiment the pharmaceutical composition can be applied to at least a portion of an outer surface of the tampon body 2. In another embodiment the pharmaceutical composition can be applied to the material from which the tampon body 2 is made prior to manufacture of the tampon 1. For example, when the tampon 1 is made from strips of hemp fiber, the pharmaceutical composition can be applied to the hemp fiber strip prior to rolling or folding the hemp fiber strip into the tampon body 2.

In addition to cannabidiol the pharmaceutical composition may contain other components that can be selected depending on the desired result. For example the pharmaceutical composition may contain components that influence the rate of release of cannabidiol during use or components that influence the consistency and physical properties of the pharmaceutical composition as a function of temperature, pH or moisture.

For example cannabidiol may be combined with biodegradable polymers including natural and synthetic polymers alone or in combination. Non-limiting examples of such polymers are polysaccharides such as alginate, dextran, cellulose, collagen, and chemical derivatives thereof, proteins such as albumin and gelatin and copolymers and blends thereof, polyhydroxy acids such as polylactides, polyglycolides and co-polymers thereof, poly ethylene terephthalate, polybutyric acid, polyvaleric acid, polylactide-co-caprolactone, polyanhydrides, polyorthoesters, and blends and co-polymers thereof.

The pharmaceutical composition may also comprise a hydrophobic material such as a solidifying agent, wax, solid ester, solid fatty alcohol or acid, hydrogenated vegetable oil, cocoa oil, solid triglycerides, natural soft solid materials (i.e., cocoa butter), solid alkyl silicones, and the like, which allows gradual diffusion of cannabidiol from the hydrophobic material to the body of the subject, while preventing loss of the cannabidiol during flow of body fluids. In one embodiment, the solidifying agent can be solid at room temperature but can soften at body temperature to increase the release rate of cannabidiol once the product has been in contact with the body for a period of time.

In an embodiment, the pharmaceutical composition comprises cannabidiol and cocoa oil. Applying the pharmaceutical composition on the tampon body 2 as a CBD containing oil allows covering at least a portion of the tampon body 2 in soothing CBD-oil. As a result, insertion of the tampon feels much smoother, reducing the likelihood of damaging the vaginal walls. In turn this also reduces the risk of Toxic Shock Syndrome (TSS)—a rare, but deadly condition associated with tampon use. TSS is caused by tampon insertion resulting in minor incisions to the vaginal walls, which then allow for bacteria to enter the user's bloodstream directly.

In yet another embodiment the pharmaceutical composition consists exclusively of cannabidiol and cocoa oil. A mixture consisting of cannabidiol and cocoa oil can advantageously be prepared by mixing CBD isolate in the form of CBD crystal powder with cocoa oil. The mixture consisting of cocoa and CBD can advantageously be applied to the tampon body 2 using a rolling brush.

In another embodiment, the pharmaceutical composition can comprise cannabidiol, cocoa oil and beeswax. Advantageously, the ratio of beeswax:oil in the pharmaceutical composition is preferably 1:2, further preferably 1:3, further preferably 1:4, further preferably 1:5. The ratio of beeswax to oil is preferably selected so that a gum-like consistency of the pharmaceutical composition results. Such a consistency advantageously allows applying the pharmaceutical composition on the surface of the tampon body 2, for example with a syringe or other applicator, in discrete patterns, such as spiral or straight strip patterns, while still ensuring dissolution of the pharmaceutical composition upon insertion of the tampon into the vagina and release of the cannabidiol.

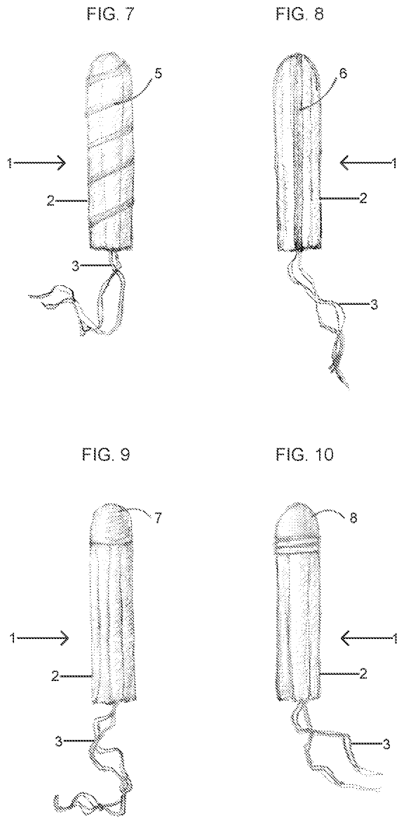

FIGS. 7 and 8 illustrate application of the pharmaceutical composition containing cannabidiol on the surface of the tampon body 2 in different configurations. In FIG. 7 the pharmaceutical composition is applied as a strip 5, which extends spirally on the surface of the tampon body 2 along the longitudinal axis L of the tampon body 2. In FIG. 8 the pharmaceutical composition is applied on the surface of the tampon body 2 as a strip 6 extending in a straight line from the proximal end 2.1 of the tampon body 2 toward the distal end 2.2 of the tampon body 2. Any other patterns of the pharmaceutical composition on the surface of the tampon body 2 are also possible such as radially extending strips or a grid pattern and are considered to be within the scope of some embodiments.

FIGS. 3 and 4 show another example of providing the tampon 1 with the pharmaceutical composition. In the embodiment shown in FIGS. 3 and 4 the pharmaceutical composition is provided on the tampon body 2 in the shape of a cup 4 that covers the proximal end 2.1 of the tampon body 2 and the circumference of the tampon body 2 along a longitudinal section of the tampon body 2. A cross sectional view of this embodiment is shown in FIG. 6.

FIG. 9 shows yet a further example of providing the tampon with the pharmaceutical composition. In FIG. 9 the pharmaceutical composition is provided by way of a tip 7 placed on the proximal end 2.1 of the tampon body 2.

With reference to FIGS. 3, 4 and 9, the cup 4 and the tip 7 may be configured as a container that encloses a space in which the pharmaceutical composition is contained. In this case the container may be made of a different material than the pharmaceutical composition. The cup 4 and the tip 7 may also be comprised entirely of the pharmaceutical composition, i.e., in this case the pharmaceutical makes up the entire cup or tip itself, instead of being contained in a space enclosed by the cup 4 or tip 7.

When the pharmaceutical composition is provided by way of a container in which the pharmaceutical composition is contained, for example in the form of the cup 4 or the tip 7, the material of the container may be selected so that the material of the container disintegrates, dissolves or becomes otherwise permeable for the pharmaceutical composition upon or during use of the tampon, resulting in release of the pharmaceutical agent into the vaginal cavity. For example, the cup 4 or tip 7 may be made of or comprise a material that is solid at room temperature and liquid at body temperature such as cocoa butter. Another example is a water-soluble material such as gelatin, which is stable in the absence of water but becomes solubilized upon contact with water-containing bodily fluids in the vagina.

The cup 4 or tip 7 can be fixed to the end of the tampon for example by using a water soluble disintegrable adhesive. Non-limiting examples of water soluble adhesives that may be advantageously used for this purpose include polyvinyl pyrrolidone, polyvinyl alcohol and polyoxazoline.

In another advantageous embodiment, the pharmaceutical composition can be applied to the tampon body 2 by dipping or immersing the tampon body 2 in a bath having the pharmaceutical composition. For this purpose the pharmaceutical composition may comprise cocoa butter, so that the pharmaceutical composition is solid at room temperature and liquid at a temperature above room temperature. The proximal end of the tampon body 2 may be dipped in the bath at the temperature at which the pharmaceutical composition is liquid and then be kept at room temperature to let the pharmaceutical composition solidify on the tampon body 2.

According to an embodiment, the amount of cannabidiol in the pharmaceutical composition is preferably in the range from 1-90 weight % cannabidiol, further preferably from 10-80 weight %, further preferably from 20-60 weight %, further preferably from 10-20 weight %, further preferably from 20-30 weight %, further preferably from 30-40 weight %, further preferably from 40-50 weight %, further preferably from 50-60 weight %, further preferably from 60-70 weight %, further preferably from 70-80 weight %, further preferably from 80-90% weight %, further preferably from 90-100 weight %. Currently particularly preferred is an amount of 70 weight % of cannabidiol in the pharmaceutical composition. According to an embodiment the amount of coca oil in the pharmaceutical composition is 10-20 weight %, 20-30 weight %, 30-40 weight %, 40-50 weight %, 50-60 weight %, 60-70 weight %, 70-80 weight %, 80-90 weight %, or 90-100 weight %. In a particularly preferred embodiment a total amount of 70 mg of the pharmaceutical composition is applied on each tampon, wherein the pharmaceutical composition consists of approximately 50 mg CBD and approximately 20 mg cocoa oil. In an embodiment, the amount of CBD in the pharmaceutical composition is 10-20 mg, 20-30 mg, 30-40 mg, 40-50 mg, 50-60 mg, 60-70 mg, 70-80 mg, 90-100 mg, 10-30 mg, 30-50 mg, 40-60 mg, 50-70 mg, 60-80 mg, 70-90 mg, or 80-100 mg. In an embodiment, the amount of cocoa oil in the pharmaceutical composition is 5-10 mg, 10-15 mg, 15-20 mg, 20-25 mg, 25-30 mg, 1-10 mg, 10-20 mg, 15-25 mg, or 20-30 mg.

FIG. 10 shows an embodiment of tampon 1 including a cellulose capsule 8 on the proximal end with rings for vacuum attachment to the tampon body. The cellulose capsule includes a hollow chamber internally, which includes a pharmaceutical composition comprising CBD.

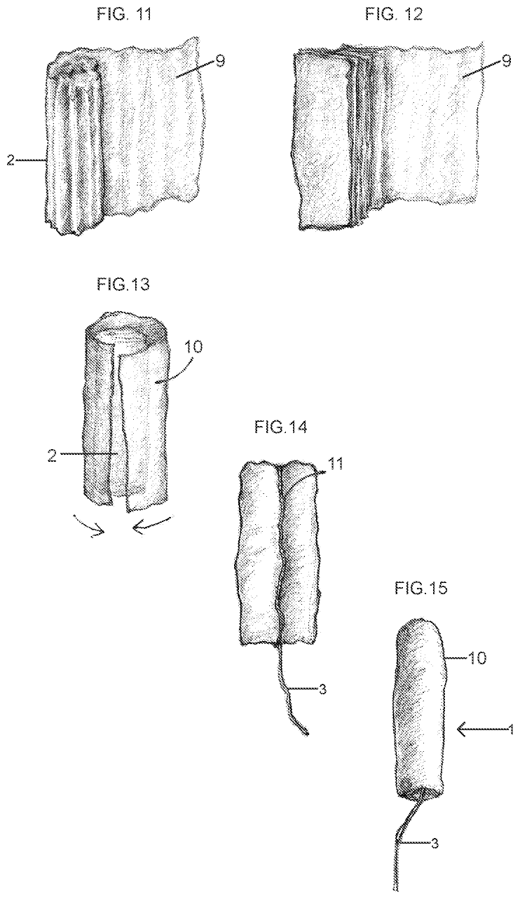

Referring to FIGS. 11-15, these Figures illustrate methods of manufacturing the tampon 1 according to one embodiment. The tampon body 2 may be formed from industrial hemp fiber strips 9. FIG. 11 illustrates a method for producing the tampon body 2 from a hemp fiber strip 9 by rolling the hemp fiber strip about a longitudinal axis of the tampon body. FIG. 12 illustrates a method for producing the tampon body 2 from a hemp fiber strip 9 by folding.

FIGS. 13-15 show the placement of a protective sock 10 on the tampon body 2. The protective sock 10 is also made of industrial hemp fibers and covers the surface of the tampon body 2. The protective sock 10 is placed around the tampon body 2 so that the ends of the protective sock 10 face each other as illustrated in FIG. 13. The ends of the protective sock 10 are then sewn together by a seam 11 as shown in FIG. 14. The protective sock is made of hemp fibers having a thicker weave than the weave of the hemp fibers of tampon body 2.

FIG. 15 shows the finished tampon 1 compressed into shape and covered by protective sock 10. The protective sock prevents hemp fibers lost from the tampon body 2 from entering the vagina. The protective sock is woven thickly to prevent passage of hemp fibers detached from the tampon body 2.

### EXAMPLES

### Example 1: Clinical Study of Tampon with CBD

The following study was performed to assess the safety and efficacy of a tampon covered with CBD oil.

Patients and Samples. A total of 130 women aged between 18 and 42 with a history of mild or sever dysmenorrhea were recruited for this study. The inclusion criteria for the patients were having a regular menstruation cycle, having been screened for STIs and demonstrated negative results, and are currently experiencing dysmenorrhea.

Study design. The study had a randomized, controlled, crossover design and ran for two consecutive menstrual cycles. Patients were divided into two groups. One group used a hemp, CBD-infused tampon. The tampon contained 50 ml of CBD oil. The tampon consisted of highly absorbent organic hemp fibers and organic hemp overwrap, organic cotton stitching thread and organic cotton removal cord, and a layer of CBD oil sprayed on top of the hemp overwrap. A second group used a standard off-the-shelf tampon product as a control. The off-the-shelf tampon consisted of blended rayon and viscose fibers with a non-woven rayon overwrap, cotton stitching thread and removal cord.

In a first visit, a patient underwent a gynecological exam and had blood drawn to test for STIs. If the patient fit inclusion criteria, in a second visit, the patient was divided into an experimental group or control group. A control test for levels of CBD in the bloodstream was performed on all participants and the results recorded. All participants were given diaries and instructed to write a detailed account of the comfort of tampon insertion and removal, comfort of wear, and levels of pain experienced.

In a third visit, at the end of the first menstrual period, patients returned to hand in their diaries. They were given a fresh batch of experimental or control tampons and new diaries.

In a fourth visit, at the end of their second menstrual period, patients returned for a full gynecological exam and blood tests to determine the level of CBD in the bloodstream.

### Results

65 patients were assigned to the experimental group and 65 patients were assigned to the control group. Of these, 61 in the experimental group and 64 patients in the control group completed the study. In the experimental group, nine out of ten participants reported experiencing no menstrual leaks and an absorption level similar to that of a super absorbent off-the-shelf tampon from a mini-sized hemp tampon. Eight out of ten participants reported experiencing a pain-relieving effect within the first 20 minutes of inserting the tampon.

Questionnaire and diary data indicated consistently positive assessments of both the experimental and control tampons, however there was a more favorable overall product rating for the experimental tampon (p=0.03). When assessing the experimental tampon, 92% of subjects rated it as good to excellent overall, with positive to neutral comfort ratings given by 95% of the subjects. Comparatively, for the control tampon, 88-89% of ratings were good to excellent and 94-95% of ratings were positive to neutral in comfort ratings.

There were low instances of reported menstrual-related symptoms such as discomfort during insertion, wear or removal (≤2.8%), and vaginal symptoms such as burning, stinging, or itching (≤1.7%).

1. A tampon for delivering a therapeutic agent, comprising:
an absorbent tampon body made of fiber;
said tampon body being provided with a pharmaceutical composition comprising an effective amount of cannabidiol, the amount of cannabidiol in the range from 2-200 mg, wherein the pharmaceutical composition further comprises a solidifying agent that softens at human body temperature, and wherein the pharmaceutical composition is applied on a surface of the tampon body.

2. The tampon of claim 1 for delivering a therapeutic agent, further comprising:
a strip comprising the effective amount of cannabidiol, the amount of cannabidiol in the range from 2-200 mg, the strip positioned on the tampon body.

3. The tampon of claim 1 for delivering a therapeutic agent, further comprising:
a cylindrical cup that encapsulates the pharmaceutical composition
wherein the cup surrounds a circumference of a proximal end of the tampon body.

4. (canceled)

5. The tampon of claim 1, wherein the amount of cannabidiol is in the range from 2-200 mg.

6. The tampon of claim 1, wherein the pharmaceutical composition consists of cocoa oil and cannabidiol.

7. The tampon of claim 1, wherein the pharmaceutical composition is applied on a surface of the tampon body.

8. The tampon of claim 1, wherein the pharmaceutical composition is applied on the tampon body in the shape of a cup surrounding a circumference of a proximal end of the tampon body.

9. (canceled)

10. (canceled)

11. The tampon of claim 1 for delivering a therapeutic agent, wherein the tampon body has longitudinal channels disposed along the length of the tampon body.

12. The tampon of claim 11 for delivering a therapeutic agent, wherein the channels are disposed diagonally from a proximal end of the tampon body and a distal end of the tampon body.

13. The tampon of claim 1 for delivering a therapeutic agent, wherein the pharmaceutical composition further comprises beeswax oil.

14. The tampon of claim 2 for delivering a therapeutic agent, wherein the strip is radially extends around the surface of the tampon body.

15. The tampon of claim 2 for delivering a therapeutic agent, wherein the strip extends spirally on the surface of the tampon body from a distal end of the tampon body to a proximal end of the tampon body.

16. The tampon of claim 2 for delivering a therapeutic agent, wherein the strip is longitudinally disposed in a straight line from a first distal end of the tampon body over a proximal end of the tampon body to a second distal end of the tampon body, the first distal end being opposite the second distal end.

17. The tampon of claim 2 for delivering a therapeutic agent, wherein the cup is comprised of a material that is solid a room temperature and liquid at human body temperature.

18. The tampon of claim 17, wherein the cup is fixed to the tampon body with a water soluble adhesive.

19. The tampon of claim 1 for delivering a therapeutic agent, further comprising:
a sock of a fiber-based material of thicker weave than the weave of fibers of the tampon body, the sock removably attached to the tampon body.

20. The tampon of claim 1 for delivering a therapeutic agent, further comprising:
a cellulose capsule on a proximal end of the tampon body, the capsule having rings for vacuum attachment to the tampon body and the capsule having a hollow chamber.
